(19) United States
(12) Patent Application Publication
(10) Pub. No.: US 2022/0001364 A1
(43) Pub. Date: Jan. 6, 2022

(54) MFI TOPOLOGICAL STRUCTURE SILICON MOLECULAR SIEVE, PREPARATION METHOD THEREOF AND CATALYST CONTAINING THE SAME

(71) Applicant: ZHEJIANG HENGLAN SCIENCE & TECHNOLOGY CO., LTD., Hangzhou (CN)

(72) Inventors: Shibiao CHENG, Hangzhou (CN); Songlin WANG, Hangzhou (CN); Fei SHEN, Hangzhou (CN); Han WANG, Hangzhou (CN); Yixuan HE, Hangzhou (CN); Zhaobin JIANG, Hangzhou (CN); Zhimin HU, Hangzhou (CN)

(73) Assignee: ZHEJIANG HENGLAN SCIENCE & TECHNOLOGY CO., LTD., Hangzhou (CN)

(21) Appl. No.: 17/208,535

(22) Filed: Mar. 22, 2021

(30) Foreign Application Priority Data
Jul. 3, 2020 (CN) 202010636836.6
Jul. 3, 2020 (CN) 202010636974.4

(51) Int. Cl. B01J 29/76; C07D 201/04; B01J 29/78; C01B 39/48; B01J 35/02; B01J 21/08; B01J 37/04; B01J 37/08; C07D 223/10; B01J 29/70; B01J 35/10; B01J 37/00; B01J 37/06

(52) U.S. Cl. CPC B01J 29/76; C01P 2002/72; B01J 29/78; C01B 39/48; B01J 35/023; B01J 21/08; B01J 37/04; B01J 37/08; C07D 223/10; B01J 29/7049; B01J 35/1019; B01J 37/009; B01J 35/1014; B01J 37/0045; B01J 37/06; C01P 2004/61; C01P 2004/62; C01P 2004/04; C07D 201/04

(57) ABSTRACT

The present disclosure discloses a MFI topological structure silicon molecular sieve, a preparation method thereof and a catalyst containing the MFI topological structure silicon molecular sieve, wherein the molecular sieve containing a silicon element, an oxygen element and a metallic element, the ions of said metallic element have a Lewis acid characteristic; the content of the metallic element in the molecular sieve is within a range of 5-100 μg/g based on the total amount of the molecular sieve; the BET specific surface area of the molecular sieve is within a range of 400-500 m²/g.

### CROSS REFERENCE TO RELATED APPLICATIONS

The application claims priorities to the Chinese Application No. 202010636836.6, filed on Jul. 3, 2020, entitled “MFI Topological Structure Silicon Molecular Sieve And Preparation Method And Application Thereof”, and the Chinese Application No. 202010636974.4, filed on Jul. 3, 2020, entitled “Catalyst Containing MFI Topological Structure Silicon Molecular Sieve And Preparation Method And Application Thereof, And Gas Phase Beckmann Rearrangement Reaction Method”, both of which are specifically and entirely incorporated herein by reference.

### FIELD

The present disclosure relates to the field of preparing silicon molecular sieves, in particular to a MFI topological structure silicon molecular sieve, a preparation method thereof, and a catalyst containing the MFI topological structure silicon molecular sieve.

### BACKGROUND

Silicalite-1 molecular sieve, also known as all-silica molecular sieve, pure-silica molecular sieve, and silicon molecular sieve, was first successfully synthesized in 1978 by E. M. Flanigen, et al. of the Union Carbide Corporation of the United States of America (USA), was one of the members of the “Pentasil” family. The silicon molecular sieve is an aluminum-free molecular sieve with a MFI topological structure, it is the molecular sieve with the simplest ingredients in a ZSM-5 type structure molecular sieve family, its skeleton is only consisting of silicon atoms and oxygen atoms, and the basic structural unit is SiO₄ tetrahedron. The silicon molecular sieve having a MFI topological structure is provided with rich micro-porous structure as well as regular and uniform three-dimensional pore channels, has the tangible crystal structure of a ZSM-5 type molecular sieve, higher internal specific surface area, desirable thermal stability, adsorption capacity and desorption capacity and other properties. The development and application of silicon molecular sieves in the fields of membrane adsorption separation, purification, catalytic materials and the like have attracted increasing attention of the industry insiders.

The silicon molecular sieve can be used as a material for membrane separation and a catalyst for producing caprolactam through gas phase Beckmann rearrangement reaction of cyclohexanone oxime. However, the silicon molecular sieve synthesized by the prior art has a high content of amorphous silicon oxide, poor relative crystallinity and larger crystal particles.

U.S. Pat. No. 4,061,724A discloses a silicon molecular sieve, which is prepared from raw materials containing no aluminum source, only containing silicon source, alkali source, template agent and water; different from a silicon molecular sieve formed by extracting the skeleton aluminum, the silicon molecular sieve is directly synthesized and has a MFI topological crystal structure. The silicon source used in the silicon molecular sieve is one of silica sol, silica gel or white carbon black, and the silica source is synthesized from the reaction mixture consisting of H₂O, SiO₂, M₂O and Q₂O in a molar ratio of 150-700:13-50:0-6.5:1 by hydrothermal crystallization at the temperature of 100-250° C. and autogenous pressure for 50-150 hours, wherein M is an alkali metal, Q is quaternary cation with the molecular formula of R₄X⁺, R represents hydrogen or an alkyl with 2-6 carbon atoms, and X is phosphorus or nitrogen.

JP59164617A discloses a MFI structure silicon molecular sieve, which is prepared by using ethyl orthosilicate as a silicon source, tetrapropylammonium hydroxide as a template agent and an alkali source.

CN102050464A discloses a method for synthesizing a silicon molecular sieve, the method comprises the following steps: (1) mixing ethyl orthosilicate and tetrapropylammonium hydroxide at room temperature, stirring, fully hydrolyzing, and adding water to form a mixture having the molar composition of TPAOH/SiO₂=0.05-0.5, EtOH/SiO₂=4, H₂O/SiO₂=5-100; (2) crystallizing the mixture in an airtight reaction kettle under autogenous pressure, subsequently subjecting to filtering, washing, drying, and roasting at 400-600° C. for 1-10 hours to obtain the silicon molecular sieve.

Caprolactam is generally obtained through a Beckmann rearrangement reaction of cyclohexanone oxime, the gas phase Beckmann rearrangement reaction of cyclohexanone oxime performed by using a solid acid catalyst is a new process for realizing thioammonium-free production of caprolactam, the new process has the advantages of avoiding equipment corrosion and environmental pollution, free of byproduct ammonium sulfate and the like, and the separation and purification of products are greatly simplified, thus the gas phase Beckmann rearrangement reaction process without ammonium sulfate has attracted great attention from the industry insider.

In order to develop a solid acid catalyst suitable for the gas phase Beckmann rearrangement reaction, researchers in China and foreign countries have conducted a great deal of researches and explorations. As disclosed in EP576295, a molecular sieve is formed into microspheres by spray drying without adding any binder, and the microspheres are then subjected to heat treatment in water such that the microspherical catalyst can be used in the reaction of converting cyclohexanone oxime into caprolactam. Although the catalyst shows a certain degree of activity, the activity strength of the catalyst cannot meet the requirement of industrial application, and the catalyst is prone to deactivate and has short service life and cannot meet the requirement of industrialization.

However, when the silicon molecular sieve synthesized by the prior art is used as a catalyst in the gas phase Beckmann rearrangement reaction of cyclohexanone oxime, the improvement effects of the cyclohexanone oxime conversion rate and the caprolactam selectivity are not obvious, and the silicon molecular sieve does not match with the whole process of the gas phase Beckmann rearrangement reaction of cyclohexanone oxime, and the economic efficiency of industrial production needs to be further improved, thus it is necessary to develop a new silicon molecular sieve and a novel catalyst containing the same.

### SUMMARY

The present disclosure aims to solve the problems of low cyclohexanone-oxime conversion rate and undesirable caprolactam selectivity in the prior art, and provides a MFI topological structure silicon molecular sieve, a preparation method thereof and a catalyst containing the MFI topological structure silicon molecular sieve. When the catalyst prepared with the molecular sieve is applied to the gas phase Beckmann rearrangement reaction of cyclohexanone oxime, the catalyst has the characteristics of higher cyclohexanone-oxime conversion rate and caprolactam selectivity.

For the sake of accomplishing the aim, a first aspect of the present disclosure provides a MFI topological structure silicon molecular sieve comprising a silicon element, an oxygen element and a metallic element, wherein the ions of said metallic element have a Lewis acid characteristic; the content of the metallic element in the molecular sieve is within a range of 5-100 μg/g based on the total amount of the molecular sieve; the BET specific surface area of the molecular sieve is within a range of 400-500 m²/g.

The content of the metallic element in the molecular sieve is preferably within a range of 6-90 μg/g, and further preferably 30-80 μg/g.

Preferably, the metallic element has an ionic valence state of +3 and/or an ionic valence state of +4.

In a second aspect, the present disclosure provides a method for preparing a MFI topological structure silicon molecular sieve, the method comprises the following steps:

(1) Mixing ethyl orthosilicate, ethanol, metal source, tetrapropylammonium hydroxide with water to obtain a colloid mixture; wherein the molar ratio of ethyl orthosilicate calculated by SiO₂, ethanol, tetrapropylammonium hydroxide and water is 1: (4-25):(0.06-0.45): (6-100); the weight ratio of the ethyl orthosilicate calculated by SiO₂ relative to the metal source calculated by metallic element is (10000-200000): 1;

(2) Subjecting the colloid mixture to a two-stage crystallization with an ethanol-hydrothermal system under variable temperatures, wherein the conditions of the two-stage crystallization with an ethanol-hydrothermal system under variable temperatures comprise: crystallizing at 40-80° C. for 0.5-5 days, and then crystallizing at 80-130° C. for 0.5-5 days;

(3) Subjecting the crystallization mother liquor obtained in the step (2) to filtering and roasting sequentially to obtain a molecular sieve;

The ions of the metallic element in the metal source have a Lewis acid characteristic.

Preferably, the molar ratio of ethyl orthosilicate calculated by SiO₂, ethanol, tetrapropylammonium hydroxide and water is 1:(4-15):(0.06-0.3):(15-50).

Preferably, the weight ratio of the ethyl orthosilicate calculated by SiO₂ relative to the metal source calculated by metallic element is (10,000-100,000): 1.

In a third aspect, the present disclosure provides a catalyst comprising a MFI topological structure silicon molecular sieve, wherein the catalyst comprising a molecular sieve and a binder; the content of the molecular sieve based on the dry weight in the catalyst is 50-95 wt %, and the content of the binder in terms of oxide is 5-50 wt %, based on the dry weight of the catalyst;

the molecular sieve comprises metallic element, the ions of the metallic element have a Lewis acid characteristic; the content of the metallic element in the molecular sieve is 5-100 μg/g based on the total amount of the molecular sieve.

It is believed in the prior art that in terms of the MFI topological structure molecular sieve, the molecular sieve with a high Si/Al ratio is conducive to the proceeding of the gas phase Beckmann rearrangement reaction, the nearly neutral silicon hydroxyl is the active center of the gas phase Beckmann rearrangement reaction, and the acid site formed by the metal-O—Si is the active center of the side reaction, which is not beneficial to the proceeding of the gas phase Beckmann rearrangement reaction. Therefore, it is considered that during a process of synthesizing a silicon molecular sieve, the metal ions having a Lewis acid characteristic will affect the Beckmann rearrangement reaction and cause the increased side reactions, thus the metallic element whose ions have a Lewis acid characteristic is not generally added. The inventors of the present disclosure have discovered that in the process of preparing the molecular sieve, the addition of a trace amount of metallic element whose ions have a Lewis acid characteristic is conducive to improving stability of the molecular sieve catalyst, when the molecular sieve is applied to the gas phase Beckmann rearrangement reaction of cyclohexanone oxime, the cyclohexanone-oxime conversion rate and caprolactam selectivity are higher.

In the method of preparing molecular sieve provided by the present disclosure, ethanol is used, and a trace amount of metal with Lewis acid characteristic, particularly the metal with an ionic valence state of +3 and/or +4 (the preferable embodiment enables metal ions to enter a molecular sieve skeleton more easily and the charges can be balanced more easily) is simultaneously added, and a two-stage crystallization with an ethanol-hydrothermal system under variable temperatures is performed to obtain the molecular sieve having a MFI topological structure and containing the metal ions with Lewis acid characteristic. When the molecular sieve is applied in the production of caprolactam, it can increase the conversion rate of cyclohexanone-oxime and the selectivity of caprolactam, and improve economic efficiency of a novel gas phase Beckmann rearrangement process technology.

In addition, the ethanol is adopted in the preparation process of the MFI topological structure silicon molecular sieve, the ethanol in the preparation process of the molecular sieve can be recovered, the ethanol can be applied to the gas phase Beckmann rearrangement reaction which uses the ethanol as a reaction solvent, and can also be applied in the crystallization refining of crude caprolactam, so as to improve the caprolactam selectivity and product quality, lower the production costs and alleviate the environmental protection pressure.

### BRIEF DESCRIPTION OF THE DRAWINGS

The accompanying drawings are provided here to facilitate further understanding on the present disclosure, and constitute a part of this document. They are used in conjunction with the following embodiments to explain the present disclosure, but shall not be comprehended as constituting any limitation to the present disclosure.

FIG. 1 illustrates an X-ray diffraction spectrogram of the catalyst containing a MFI topological structure molecular sieve prepared in Example 1 of the present disclosure;

FIG. 2 is a photomicrograph (100×) of the catalyst containing a MFI topological structure molecular sieve prepared in Example 1 of the present disclosure;

FIG. 3 shows a transmission electron microscope (TEM) photograph of the MFI topological structure molecular sieve prepared in Example 1 of the present disclosure;

FIG. 4 illustrates a TEM photograph of the catalyst containing a MFI topological structure molecular sieve prepared in Example 1 of the present disclosure.

### DETAILED DESCRIPTION

The terminals and any value of the ranges disclosed herein are not limited to the precise ranges or values, such ranges or values shall be comprehended as comprising the values adjacent to the ranges or values. As for numerical ranges, the endpoint values of the various ranges, the endpoint values and the individual point value of the various ranges, and the individual point values may be combined with one another to produce one or more new numerical ranges, which should be deemed have been specifically disclosed herein.

A first aspect of the present disclosure provides a MFI topological structure silicon molecular sieve comprising a silicon element, an oxygen element and a metallic element, wherein the ions of said metallic element have a Lewis acid characteristic; the content of the metallic element in the molecular sieve is within a range of 5-100 μg/g based on the total amount of the molecular sieve; the BET specific surface area of the molecular sieve is within a range of 400-500 m²/g.

The ions of the metallic element have a Lewis acid characteristic, which means that the ions of the metallic element can accept an electron pair (i.e., duplet).

It should be noted that, the metallic element is contained in the MFI topological structure silicon molecular sieve of the present disclosure in an extreme trace amount, it can be asserted that a trace amount of the metallic element exists in the molecular sieve skeleton in the form of metal ion.

In the MFI topological structure silicon molecular sieve provided by the present disclosure, metallic element exists on the molecular sieve skeleton in the form of metal cation.

In the present disclosure, the content of the metallic element is measured by using an inductively coupled plasma (ICP) atomic emission spectrometer 7000DV, manufactured by PE (Perkin Elmer Incorporation) of the USA, under the test conditions as follows: the molecular sieve is dissolved by using HF acid or aqua regia to completely dissolve silicon oxide and metal oxide in the sample, and the content of metal ions is measured in an aqueous solution.

The present disclosure has wider selection range in regard to the contents of silicon element and oxygen element in the molecular sieve, and in a specific embodiment, the sum of the contents of the silicon element, the oxygen element and the metallic element in the molecular sieve is 100% based on the total amount of the molecular sieve.

According to the present disclosure, the BET specific surface area of the molecular sieve is preferably within a range of 420-450 m²/g. In this preferred circumstance, it is more conducive to improving the performance of the molecular sieve as a catalyst.

The present application has wider selection range in regard to the crystalline grain particle size of the molecular sieve, the crystalline grain particle size of the molecular sieve is preferably within a range of 0.1-0.3 μm, more preferably 0.1-0.25 μm, and further preferably 0.1-0.2 μm. In this preferred circumstance, it is more conducive to improving the catalytic performance of the molecular sieve as a catalyst. The crystalline grain size of the molecular sieve in the present disclosure is measured by using a field emission scanning electron microscope with a model number S-4800 manufactured by the Hitachi Corporation of Japan.

The external specific surface area of the molecular sieve can be selected from a wide range of the present disclosure, and the external specific surface area of the molecular sieve is preferably within a range of 30-60 m²/g, more preferably 35-50 m²/g. In the present disclosure, the BET specific surface area and the external specific surface area of the molecular sieve are measured with the N₂ adsorption-desorption method, specifically, measured by an automatic adsorption apparatus with a model number ASAP-2460 manufactured by the Micromeritics Instrument Corporation in the USA, under the following test conditions: N₂ is used as an adsorbate, the adsorption temperature is −196.15° C. (liquid nitrogen temperature), and degassing is performed at 1.3 Pa and the constant temperature 300° C. for 6 hours.

According to a preferred embodiment of the present disclosure, the content of the metallic element in the molecular sieve is within a range of 6-90 μg/g, preferably 30-80 μg/g, based on the total amount of the molecular sieve. Specifically, the concentration may be for example 30 μg/g, 35 μg/g, 40 μg/g, 45 μg/g, 50 μg/g, 55 μg/g, 60 μg/g, 70 μg/g, 75 μg/g, 80 μg/g, or any value in the ranges formed by any two of the numerical values. In the preferred embodiment, the molecular sieve has better catalytic performance, and is more conducive to improving the conversion rate of cyclohexanone oxime and the selectivity of caprolactam. If the content of the metallic element in the present disclosure is excessive, the Lewis acid characteristic of the molecular sieve may be enhanced, which will induce the unnecessary side reactions, hamper improvement of the caprolactam selectivity; if the content of the metallic element is deficient, it is not beneficial to prolonging the service life of the molecular sieve catalyst and enhancing the stability.

Any metallic element whose ion has a Lewis acid characteristic may be used in the present disclosure, and preferably, the metallic element is at least one selected from the group consisting of transition metallic element, group IIIA element and group IVA element.

According to the present disclosure, the transition metallic element is preferably at least one metallic element selected from the group consisting of group IB, group IIB, group IVB, group VB, group VIB, group VIIB and group VIII.

According to a preferred embodiment of the present disclosure, the metallic element is at least one element selected from the group consisting of Al, Ga, Ge, Ce, Ag, Co, Ni, Cu, Zn, Mn, Pd, Pt, Cr, Fe, Au, Ru, Rh, Ti, Zr, V, Mo and W.

Further preferably, the metallic element has an ionic valence state of +3 and/or an ionic valence state +4. The inventors of the present disclosure have discovered in the research process that the metallic element with an ionic valence state of +3 and/or an ionic valence state of +4 is more favorable for the metallic element to enter the molecular sieve skeleton and more conducive to charge balance.

According to the present disclosure, the metallic element is further preferably at least one element selected from the group consisting of Fe, Al, Ga, Ge, Cr, Ti, Zr, and Ce. Such a preferred embodiment is more beneficial to improve the performance of the molecular sieve, thereby improving the conversion rate of cyclohexanone oxime and the selectivity of caprolactam.

In a second aspect, the present disclosure provides a method for preparing a MFI topological structure silicon molecular sieve, the method comprises the following steps:

(1) Mixing ethyl orthosilicate, ethanol, metal source, tetrapropylammonium hydroxide with water to obtain a colloid mixture; wherein the molar ratio of ethyl orthosilicate calculated by SiO₂, ethanol, tetrapropylammonium hydroxide and water is 1:(4-25):(0.06-0.45):(6-100); the weight ratio of the ethyl orthosilicate calculated by SiO₂ relative to the metal source calculated by metallic element is (10,000-200,000): 1;

(2) Subjecting the colloid mixture to a two-stage crystallization with an ethanol-hydrothermal system under variable temperatures, wherein the conditions of the two-stage crystallization with an ethanol-hydrothermal system under variable temperatures comprise: crystallizing at 40-80° C. for 0.5-5 days, and then crystallizing at 80-130° C. for 0.5-5 days;

(3) Subjecting the crystallization mother liquor obtained in the step (2) to filtering and roasting sequentially to obtain a molecular sieve;

The ions of the metallic element in the metal source have a Lewis acid characteristic.

Unless otherwise specified in the present disclosure, the molar ratio and the mass ratio of the materials in the molecular sieve preparation process refer to the molar ratio and the mass ratio of the used amount when the materials are fed (charged).

According to a preferred embodiment of the present disclosure, the method for preparing the molecular sieve provided by the present disclosure does not include an addition of an organic amine. In this preferred embodiment, the molecular sieve has better properties. In the present disclosure, the organic amine refers to at least one of aliphatic amine compounds, and may be, for example, at least one of mono-n-propylamine, di-n-propylamine, tri-n-propylamine, ethylamine, n-butylamine, ethylenediamine, and hexamethylenediamine.

According to the present disclosure, a specific silicon source, a specific metal source and a specific organic template agent are adopted in combination with ethanol, so as to prepare the molecular sieve with a specific structure under the condition of specific dosage, and the molecular sieve has better catalytic performance. The molecular sieve is particularly suitable for gas phase Beckmann rearrangement reaction of cyclohexanone oxime, and is more favorable for improving the economic efficiency of the whole process.

According to a preferred embodiment of the present disclosure, the molar ratio of ethyl orthosilicate calculated by SiO₂, ethanol, tetrapropylammonium hydroxide and water is 1:(4-15):(0.06-0.3):(15-50), more preferably 1:(6-14):(0.1-0.25):(20-40). In this preferred embodiment, the produced molecular sieve has better catalytic performance.

According to a preferred embodiment of the present disclosure, the weight ratio of the ethyl orthosilicate calculated by SiO₂ relative to the metal source calculated by metallic element is (10000-100000): 1, more preferably (15000-50000): 1. Under the preferred embodiment, the more suitable amount of metal enters the skeleton of the molecular sieve, which is more beneficial to improving the catalytic performance of the molecular sieve.

According to the method provided by the present disclosure, the selection of the metallic element in the metal source is as previously mentioned, and the content is not repeated here.

The present disclosure has a wide range of choices for the metal source, which is a compound containing various metallic element and being capable of providing the metallic element, and the compound containing the metallic element is preferably soluble. In the present disclosure, the term “soluble” means that it can be dissolved in a solvent directly or in the presence of a co-solvent, and the solvent is preferably water.

According to the present disclosure, the metal source is preferably at least one selected from the group consisting of a metal nitrate, a metal chloride, a metal sulfate, a metal acetate, and an ester metal compound. In a specific embodiment, the ester metal compound is tetraethyl titanate and/or tetrabutyl titanate.

It is preferable in the present disclosure, when the metal is an Al element, the metallic aluminum source can also be a compound in the form of alumina, such as SB powder, V250 and pseudoboehmite.

According to a preferred embodiment of the present disclosure, the metal source is preferably at least one selected from the group consisting of Fe(NO₃)₃, Ni(NO₃)₂, tetrabutyl titanate, Pd(NO₃)₂, Ce(NO₃)₄, Al(NO₃)₃, Cu(NO₃)₂, ZrOCl₂, Ga(NO₃)₃, H₂PtCl₆ and Cr(NO₃)₃, and is further preferably at least one selected from the group consisting of Fe(NO₃)₃, tetrabutyl titanate, Al(NO₃)₃, Ga(NO₃)₃ and Cr(NO₃)₃. The metal source may either contain or not contain crystal water, and the present disclosure does not impose a particular limitation thereto.

The order of mixing in step (1) is not particularly limited in the present disclosure, as long as the colloidal mixture can be obtained; any two of the compounds may be initially blended and then mixed with the remaining substances, or any three of the compounds may be initially blended and subsequently mixed with the remaining substances. Preferably, it is desirable to avoid gel formation during the process of charging materials and also to prevent excessive temperature rise of the liquid phase during the process of charging materials. Specifically, for example, ethanol and tetrapropylammonium hydroxide may be blended, water and a metal source may then be added, and ethyl orthosilicate is subsequently added; or the ethanol and tetrapropylammonium hydroxide may be blended, water and tetraethoxysilane are sequentially added, a metal source is further added; or the ethyl orthosilicate, ethanol and tetrapropylammonium hydroxide are initially blended, the water and the metal source are subsequently added; alternatively, the ethyl orthosilicate, ethanol, tetrapropylammonium hydroxide are initially mixed, water is then added, and a metal source is subsequently added. In the present disclosure, the metal source may be introduced alone or in the form of a solution.

According to the present disclosure, the mixing of step (1) preferably comprises: mixing ethanol and tetrapropylammonium hydroxide, then adding ethyl orthosilicate, and further adding water and a metal source.

The present application has wide selection ranges in regard to the specific operation of the mixing process, according to a preferred embodiment of the present disclosure, and the mixing is performed under the stirring conditions. In the present disclosure, the stirring time is not particularly limited, so long as the colloidal mixture can be obtained. For example, the mixture may be stirred at normal temperature (25° C.) for 2-6 hours.

According to a preferred embodiment of the present disclosure, the conditions of the two-stage crystallization with an ethanol-hydrothermal system under variable temperatures comprise: crystallizing at 50-80° C. for 1-1.5 days, and then crystallizing at 100-120° C. for 1-3 days. Under the preferred embodiment, the utilization rate of crystallization raw materials is further improved, and the prepared catalyst containing the molecular sieve has better catalytic performance under the specific crystallization conditions. In the present disclosure, the two-stage crystallization with an ethanol-hydrothermal system under variable temperatures is preferably performed in a closed system under an autogenous pressure, for example, in an airtight reaction kettle.

In the present disclosure, the crystallization with an ethanol-water system means that the crystallization is performed under a saturated vapor pressure of a specific temperature in the co-presence of ethanol and water.

The filtration method of the present disclosure is not particularly limited, and various filtration methods conventionally used in the art may be used, as long as the purpose of solid-liquid separation can be achieved.

According to the present disclosure, it is preferable that the step (3) further comprises: washing the crystallization mother liquor before the filtering process. The washing is not particularly limited in the present disclosure, and may be any of various washing methods conventionally used in the art, and the detergent used in the washing process is not particularly limited in the present disclosure, for example, the detergent may be water.

According to the method provided by the present disclosure, it is preferable that the method further comprises: the crystallization mother liquor is subjected to ethanol removal prior to the filtration in step (3) (preferably prior to washing, if washing is also included). In the present disclosure, given that the ethanol contains organic oxygen during the industrial production, the discharge of ethanol into wastewater may result in environmental problems, thus the ethanol removal operation is required.

In the present disclosure, the conditions of ethanol removal are selected from a wide range, as long as the purpose of removing ethanol is achieved; the conditions of ethanol removal preferably comprise: the temperature is within a range of 50−90° C., preferably 60-90° C.; the time is within a range of 1-24 h, preferably 1-12 h.

Specifically, the reaction kettle may be opened after the temperature of the reaction kettle is lowered to an operable temperature, and the temperature of the reaction kettle is then raised to 50-90° C. to evaporate ethanol. In the ethanol removal operation of the present disclosure, water may be added into the reaction kettle to maintain the liquid level of the reaction kettle, which is beneficial to improving efficiency of the ethanol removal process.

According to the present disclosure, it is preferable that the step (3) further includes: the product obtained by filtration is subjected to drying after the filtering process and before the roasting process. In the present disclosure, the drying may be performed with a method existing in the art, and specifically, for example, the drying conditions may include: the temperature is within a range of 80-150° C., and the time is 2-36 h.

The present disclosure has wide selection range in regard to the roasting conditions, the roasting conditions preferably comprise: the temperature is within a range of 400-600° C., preferably 500-580° C. In the present disclosure, the roasting time may be selected from a wide range, which can be measured according to the amount of the material to be roasted; when the amount of the material to be roasted is large, the roasting time can be appropriately extended, so long as the template agent (it refers to tetrapropylammonium hydroxide in the present disclosure) is completely roasted; specifically, the roasting time may be within a range of 1-20 hours, preferably 2-10 hours.

The present disclosure also provides the MFI topological structure silicon molecular sieve prepared with the method. The preparation method provided by the present disclosure enables the metal ions with Lewis acid characteristic to enter a molecular sieve skeleton structure, if the crystalline grains in the prepared molecular sieve are finer and more uniform, the catalytic performance is better; when the molecular sieves are applied to the gas phase Beckmann rearrangement reaction of cyclohexanone oxime, it has higher cyclohexanone oxime conversion rate and caprolactam selectivity.

In a third aspect, the present disclosure provides a catalyst comprising a silicon molecular sieve with a MFI topological structure silicon molecular sieve, wherein the catalyst comprising a molecular sieve and a binder; the content of the molecular sieve based on the dry weight in the catalyst is 50-95 wt %, and the content of the binder in terms of oxide is 5-50 wt %, based on the dry weight of the catalyst;

The molecular sieve comprises metallic element, the ions of the metallic element have a Lewis acid characteristic; the content of the metallic element in the molecular sieve is 5-100 μg/g based on the total amount of the molecular sieve.

In the present disclosure, the molecular sieve in the catalyst is completely identical with the MFI topological structure silicon molecular sieve provided in the aforesaid first aspect, the content is not repeated here.

In the present disclosure, the particle size of the catalyst is selected from a wide range, the particle size of the catalyst is preferably within a range of 20-200 μm, more preferably 40-150 μm. In this preferable circumstance, it is more advantageous to enhance the stability of the catalyst and further improve the catalytic performance of the catalyst. In the present disclosure, the particle size distribution of the catalyst is measured by a 2000E type laser particle size analyzer manufactured by the Dandong Bettersize Instruments Co., Ltd., the test method is a wet process test, water is used as a medium, and the mass concentration of a sample is within a range of 0.5%-2%, the scanning speed is 2,000 times/second.

According to a preferred embodiment of the present disclosure, the catalyst has an abrasion index K less than 3%/h, preferably 0-2%/h. In this preferred embodiment, the catalyst has a higher strength, which is more advantageous for improving the catalyst stability. In the present disclosure, the lower is the abrasion index K, the higher is the abrasion resistance of the catalyst. In the present disclosure, the abrasion index K is measured on an abrasion index analyzer according to the Industry Standard RIPP29-90 in the Petrochemical Analysis Method (Cuiding Yang, et al, Science Press of China, 1990) compiled by the SINOPEC Research Institute of Petroleum Processing (RIPP).

According to a preferred embodiment of the present disclosure, the content of the molecular sieve based on the dry weight in the catalyst is 50-70 wt %, and the content of the binder in terms of oxide is 30-50 wt %, based on the dry weight of the catalyst. In this preferred embodiment, it is more conducive to increasing the conversion rate of cyclohexanone oxime and the selectivity of caprolactam.

According to a preferred embodiment of the present disclosure, the binder is silicon oxide.

The preparation method of the catalyst may be selected from a wide range, as long as the specific catalyst can be prepared; preferably, the present disclosure also provides a method for preparing a MFI topological structure silicon molecular sieve, the method comprises the following steps:

(a) Mixing ethyl orthosilicate, ethanol, metal source, tetrapropylammonium hydroxide with water to obtain a colloid mixture; wherein the molar ratio of ethyl orthosilicate calculated by SiO₂, ethanol, tetrapropylammonium hydroxide and water is 1:(4-25):(0.06-0.45):(6-100); the weight ratio of the ethyl orthosilicate calculated by SiO₂ relative to the metal source calculated by metallic element is (10000-200000): 1;

(b) Subjecting the colloid mixture to a two-stage crystallization with an ethanol-hydrothermal system under variable temperatures, wherein the conditions of the two-stage crystallization with an ethanol-hydrothermal system under variable temperatures comprise: crystallizing at 40-80° C. for 0.5-5 days, and then crystallizing at 80-130° C. for 0.5-5 days;

(c) Concentrating the crystallization mother liquor obtained in the step (b) to obtain a molecular sieve slurry;

(d) Blending the molecular sieve slurry with a binder and pulping to obtain a molecular sieve-binder slurry; subjecting the molecular sieve-binder slurry to a mist spray forming, and then roasting;

(e) Contacting the roasted product of step (d) with an alkaline buffer solution of a nitrogen-containing compound, and subsequently carrying out drying.

The ions of the metallic element in the metal source have a Lewis acid characteristic.

Unless otherwise specified in the present disclosure, the molar ratio and the mass ratio of the materials in the molecular sieve preparation process refer to the molar ratio and the mass ratio of the used amount when the materials are fed (charged).

According to a preferred embodiment of the present disclosure, the method for preparing the molecular sieve provided by the present disclosure does not include an addition of an organic amine. In this preferred embodiment, the molecular sieve has better properties. In the present disclosure, tetrapropylammonium hydroxide is used as an organic alkali and can also be used as a template agent, and an addition of the organic amine is not required. In the present disclosure, the organic amine refers to at least one of aliphatic amine compounds, and may be, for example, at least one of mono-n-propylamine, di-n-propylamine, tri-n-propylamine, ethylamine, n-butylamine, ethylenediamine, and hexamethylenediamine.

According to the present disclosure, a specific silicon source, a specific metal source and a specific organic template agent are adopted in combination with ethanol, so as to prepare the molecular sieve with a specific structure under the condition of specific dosage, and the molecular sieve has better catalytic performance. The molecular sieve is particularly suitable for gas phase Beckmann rearrangement reaction of cyclohexanone oxime, and is more favorable for improving the economic efficiency of the whole process.

In the preparation method of the catalyst of the present disclosure, the step (a) and step (b) are completely identical with the step (1) and step (2) in the preparation method of the MFI topological structure silicon molecular sieve provided in the second aspect, the content is not repeated here.

According to the present disclosure, the pH of the crystallization mother liquor in step (c) is preferably greater than 11, preferably not less than 13, for example between 13 and 14.

In the present disclosure, the crystallization with an ethanol-water system means that the crystallization is performed under a saturated vapor pressure of a specific temperature in the co-presence of ethanol and water.

The concentration mode in step (c) may be selected from a wide range of the present disclosure, as long as the purpose of increasing the solid content of the molecular sieve slurry can be achieved.

It is preferable in the present disclosure that before the concentrating process, step (c) further comprises: washing the crystallization mother liquor until the pH of the wash water for washing the crystallization product is below 9.4, preferably below 9.2, for example, the pH is within a range of 8.5-9.2. The present disclosure does not impose specific limitation in regard to the washing process, which may be any of various washing methods conventionally used in the art; in addition, the detergent used in the washing process is not particularly limited in the present disclosure, it may be water, for example. The water may be purified water, deionized water, ion exchange water, chemical water, or other water without containing anions and cations. In the present disclosure, the washing operation may be repeated, and the number of the repeated operation is not particularly defined, the repeated operation may be performed for 1-10 times, for example.

According to a preferred embodiment of the present disclosure, the crystallization mother liquor is washed with water at a temperature of 20-80° C.

According to a preferred embodiment of the present disclosure, the washing and concentration of the molecular sieve is carried out by means of membrane filtration, for example, by using a six-tube membrane. The specific operation is well-known among those skilled in the art, the content will not be repeated here.

According to the method provided by the present disclosure, it is preferable that the method further comprises: the crystallization mother liquor is subjected to ethanol removal prior to the concentrating process (preferably prior to washing, if a washing process is also included in the method) in step (c). In the present disclosure, given that the ethanol contains organic oxygen during the industrial production, the discharge of ethanol into wastewater may result in environmental problems, thus the ethanol removal operation is required.

In the present disclosure, the conditions of ethanol removal are selected from a wide range, as long as the purpose of removing ethanol is achieved; the conditions of ethanol removal preferably comprise: the temperature is within a range of 50-90° C., preferably 60-90° C.; the time is within a range of 1-24 h, preferably 1-12 h.

Specifically, the reaction kettle may be opened after the temperature of the reaction kettle is lowered to an operable temperature, and the temperature of the reaction kettle is then raised to 50-90° C. to evaporate ethanol. In the ethanol removal operation of the present disclosure, water can be added into the reaction kettle to maintain the liquid level of the reaction kettle, which is beneficial to improving efficiency of the ethanol removal process.

In the present disclosure, the solid content of the molecular sieve slurry is selected from a wide range, and preferably, the solid content of the molecular sieve slurry in step (c) is within a range of 15-40 wt %, preferably 20-35 wt %. The preferred circumstance is more conducive to improving performance of the prepared catalyst.

According to the present disclosure, the molecular sieve-binder slurry in step (d) preferably has a solid content of 10-40 wt %, preferably 10-35 wt %. It is more advantageous to carry out the mist spray forming under the preferred circumstance, such that the abrasion index of the catalyst is lower.

According to the present disclosure, it is preferable in the molecular sieve-binder slurry, the weight ratio of the molecular sieve based on the dry weight relative to the binder calculated by SiO₂ is 1:(0.05-1), preferably 1:(0.4-0.8), further preferably 1:(0.55-0.7). In the preferable circumstance, the catalyst has better performance, and it is more conducive to improving the conversion rate of cyclohexanone oxime and the selectivity of caprolactam.

In the mist spray forming process, the binder is preferably a precursor of silicon oxide. The present disclosure provides a wide selection range for the precursor of the silicon oxide, as long as the precursor can be converted into the silicon oxide through subsequent roasting. Preferably, the precursor of the silicon oxide is silica sol and/or white carbon black, and further preferably silica sol.

The silica sol and the white carbon black of the present disclosure are commercially available.

According to the present disclosure, the silica sol preferably has a SiO₂ content of 20-45 wt %, preferably 30-40 wt %.

According to the present disclosure, the silica sol may further contain sodium ions, the content of sodium ions is selected from a wide range of the present disclosure, and preferably, the content of sodium ions is not higher than 1,000 μg/g. In the preferred circumstance, it is more conducive to improving performance of the catalyst.

The mist spray forming of the present disclosure has the conventional meaning in the art. The conditions of the mist spray forming preferably cause that the particles obtained by the mist spray forming have a particle size of 20-200 μm, further preferably 40-150 μm.

According to the present disclosure, the conditions of the mist spray forming comprise: the inlet temperature is within a range of 180-240° C., preferably 200-220° C.; the outlet temperature is within a range of 80-120° C., and preferably 90-105° C. In the preferred embodiment, the catalyst has better performance, and it is more conducive to improving the conversion rate of cyclohexanone oxime and the selectivity of caprolactam.

According to the present disclosure, the roasting conditions of step (c) preferably comprise: the temperature is within a range of 200-600° C., preferably 250-550° C., and the time is within a range of 1-20 h, preferably 2-18 h.

According to the present disclosure, it is preferable that the roasting may be a multi-stage roasting, and for instance, the roasting may specifically include stage 1) and stage 2); the conditions of the phase 1) comprise: the temperature is within a range of 200-400° C., and the time is within a range of 2-10 h; the conditions of the stage 2) comprise: the temperature is within a range of 400-600° C., and the time is within a range of 2-15 h. Further preferably, the stage 1) includes a stage 1-1) and a stage 1-2), and the conditions of the stage 1-1) include: the temperature is within a range of 200-300° C., the time is within a range of 2-5 h, and the conditions of the stage 1-2) comprise: the temperature is within a range of 300-400° C., and the time is within a range of 2-5 h; the stage 2) comprises a stage 2-1) and a stage 2-2), and the conditions of the stage 2-1) comprise: the temperature is within a range of 400-500° C., the time is within a range of 2-5 h, and the conditions of the stage 2-2) comprise: the temperature is within a range of 500-600° C., and the time is within a range of 8-13 h.

According to a preferred embodiment of the present disclosure, the alkaline buffer solution of a nitrogen-containing compound comprises an ammonium salt and an alkali.

The solvent of the alkaline buffer solution of a nitrogen-containing compound may be selected from a wide range, the solvent is preferably water.

In the present disclosure, the ammonium salt is preferably ammonium nitrate and/or ammonium acetate.

According to the present disclosure, the alkali is preferably at least one selected from the group consisting of ammonia water, tetramethylammonium hydroxide, tetraethylammonium hydroxide and tetrapropylammonium hydroxide, more preferably ammonia water.

According to a preferred embodiment of the present disclosure, the ammonium salt is contained in an amount of 0.1-20 wt %, preferably 0.5-15 wt %; the alkali is contained in an amount of 5-30 wt %, preferably 10-28 wt %.

According to the present disclosure, it is preferable that the alkaline buffer solution of a nitrogen-containing compound has a pH within a range of 8.5-13.5, preferably 10-12, and more preferably 11-11.5.

The present disclosure has wide selection range of the dosage of the alkaline buffer solution of a nitrogen-containing compound, and the alkaline buffer solution of a nitrogen-containing compound is used in an amount of 500-1,500 parts by weight, preferably 700-1,200 parts by weight, relative to 100 parts by weight of the roasted product on a dry basis.

According to the present disclosure, it is preferable that the contacting conditions comprise: the temperature is within a range of 50−120° C., preferably 70-100° C.; the pressure is within a range of 0.5-10 kg/cm², preferably 1.5-4 kg/cm²; the time is within a range of 0.1-5 h, preferably 1-3 h. In the present disclosure, the contacting process is preferably performed under stirring conditions. The stirring speed is not particularly limited in the present disclosure, and it may be appropriately selected by those skilled in the art according to the actual situation.

According to the method provided by the present disclosure, the contacting process may be subjected to repetitive operation. The number of repetitions is not particularly limited in the present disclosure, it may be determined according to the effect of the contacting process; in order to improve the performance of the catalyst, for example, the contacting process may be may be repeated for 1-3 times.

The present disclosure does not impose specific definition in regard to the conditions for drying the product prepared by contacting the product obtained from the roasting process with the alkaline buffer solution of a nitrogen-containing compound, the drying process may be performed with any means known in the prior art, as long as the solvent is removed, and the drying method includes, but is not limited to, natural drying, heat drying, and forced air drying, and specifically, for example, the drying temperature may be within a range of 100−120° C., and the drying time may be within a range of 2-36 hours.

According to the present disclosure, it is preferable that step (e) may further comprises: prior to the drying, sequentially filtering and washing the substances obtained after the roasted product obtained in step (d) is contacted with the alkaline buffer solution of a nitrogen-containing compound. The detergent used in the washing process of the present disclosure is not particularly limited, for example, the detergent may be water. Specifically, the washing process may include: washing until the pH of the filtration clear solution is within a range of 9-10.5.

The present disclosure also provides a catalyst containing a MFI topological structure silicon molecular sieve prepared with the method. The catalyst produced through the preparation method provided by the present disclosure enables the metal ions with a Lewis acid characteristic to enter a molecular sieve skeleton, and the catalyst has higher strength and desirable performance, and is particularly suitable for gas phase Beckmann rearrangement reaction of cyclohexanone oxime.

Therefore, the present disclosure further provides an application of the catalyst containing a MFI topological structure silicon molecular sieve in the gas phase Beckmann rearrangement reaction of cyclohexanone oxime. When the catalyst containing the molecular sieve provided by the present disclosure is used in the gas phase Beckmann rearrangement reaction of cyclohexanone oxime, the conversion rate of cyclohexanone oxime and the selectivity of caprolactam are higher, and the service life of the catalyst can be prolonged, thereby improving the economic efficiency of a novel gas phase Beckmann rearrangement process technology.

The present disclosure also provides a method for gas phase Beckmann rearrangement reaction of cyclohexanone oxime, the method comprises the following steps: cyclohexanone oxime is contacted with a catalyst to carry out reaction under the condition of gas phase Beckmann rearrangement reaction of cyclohexanone oxime and in the presence of a solvent, wherein the catalyst is the catalyst containing the MFI topological structure silicon molecular sieve provided by a third aspect of the present disclosure.

According to the present disclosure, the solvent is preferably ethanol. More preferably, at least a part of the ethanol is obtained in step (c) of the preparation method of the catalyst containing the MFI topological structure silicon molecular sieve provided by the present disclosure, and further preferably, the ethanol is recovered by evaporating the solution obtained by crystallization. The adoption of the preferred embodiment is more beneficial to improving the economic efficiency of the novel gas phase Beckmann rearrangement process technology. The recovery process is not particularly limited in the present disclosure, and specifically, for example, the solution obtained by the crystallization may be subjected to evaporation (preferably at a temperature of 60-90° C.) and an appropriate amount of water is supplemented during the evaporation process, so as to obtain hydrous ethanol, the hydrous ethanol may be then subjected to distillation dehydration, membrane filtration dehydration and/or molecular sieve adsorption dehydration. In the present disclosure, the distillation dehydration may be performed with any of the existing techniques in the art. The membrane filtration dehydration is not particularly limited in the present disclosure, for example, it may be performed by using a six-tube membrane. The specific operation is well known among those skilled in the art and will not be described herein. The molecular sieve adsorption dehydration is not particularly defined in the present disclosure, it may be an existing operation in the art, and the present disclosure does not repeat the details herein.

Specifically, the ethanol obtained in the preparation of the catalyst may be subjected to distillation dehydration, membrane filtration dehydration and/or molecular sieve adsorption dehydration, and then be used as a solvent for gas phase Beckmann rearrangement reaction. Taking a caprolactam production facility with a production capacity of 100,000 ton/year as an example, the production facility consumes 300 tons of ethanol as a reaction solvent and about 35 tons of catalyst used for the gas phase Beckmann rearrangement reaction every year. About 30 tons of molecular sieves are required to be used in the preparation process of about 35 tons of catalyst used for the gas phase Beckmann rearrangement reaction, and about 120 tons of ethanol can be recovered in the preparation process of about 30 tons of molecular sieve. Therefore, the recovered ethanol is used as the gas phase Beckmann rearrangement reaction solvent, the arrangement not only significantly reduces the production cost (about 40% of the solvent cost is saved), but also decreases the discharge amount of pollutants (during the molecular sieve preparation process in the art, when the slurry following the crystallization process is subjected to washing and filtering, the filtrate obtained from the filtering process is directly discharged into water).

According to the present disclosure, the gas phase Beckmann rearrangement reaction of cyclohexanone oxime is preferably carried out under an inert atmosphere. In the present disclosure, the inert atmosphere is provided by an inert gas, the inert gas is preferably at least one selected from the group consisting of nitrogen gas, helium gas, argon gas and neon gas, more preferably nitrogen gas.

According to the present disclosure, the molar ratio of the inert gas relative to the cyclohexanone oxime is preferably 1-10:1, preferably 1-5:1, more preferably 2-3:1. Such a preferable circumstance is more conducive to reducing the energy consumption, thereby improving the economic efficiency of the overall process of the gas phase Beckmann rearrangement reaction of cyclohexanone oxime. The catalyst prepared by mist spray forming has better effect by matching with the specific fluidized bed gas phase Beckmann rearrangement reaction process, such that the economic efficiency of the process is further improved.

According to a preferred embodiment of the present disclosure, the conditions of the cyclohexanone oxime gas phase Beckmann rearrangement reaction comprise: the reaction temperature is within a range of 300−500° C., preferably 350-400° C.; the reaction pressure is 0.05-0.8 MPa, preferably 0.1-0.5 MPa in terms of the gauge pressure, and the cyclohexanone oxime weight hourly space velocity is 0.1-20 h⁻¹, preferably 3-8 h⁻¹, more preferably 3-6 h⁻¹.

According to the present disclosure, it is preferable that cyclohexanone oxime accounts for 20-50 wt % of the sum of cyclohexanone oxime and solvent (preferably ethanol).

According to the present disclosure, it is preferable that the method further comprises: mixing cyclohexanone oxime with water (preferably in a molar ratio of 1:0.01-2.5), and then contacting with the catalyst in the presence of the solvent so as to perform a gas phase Beckmann rearrangement reaction. The adoption of the preferred embodiment is more beneficial to improving the stability of catalyst and the selectivity of caprolactam.

According to the present disclosure, it is preferable that the method further comprises: mixing a solution of cyclohexanone oxime and ethanol with water (preferably, water is added in an amount of 0.1-1 wt % based on the weight of a solution of cyclohexanone oxime and ethanol), and then contacting the mixture with the catalyst to perform a vapor phase Beckmann rearrangement reaction. The adoption of the preferred embodiment is more beneficial to improving the stability of catalyst and the selectivity of caprolactam.

The catalyst prepared by the present disclosure may be applied in a gas phase Beckmann rearrangement reaction of cyclohexanone oxime process under the specific process conditions, and it is beneficial to further improving the economic efficiency of the whole process. According to the present disclosure, ethanol is adopted in the preparation process of the catalyst containing the molecular sieve, so that the prepared catalyst has better performance, the catalyst is particularly suitable for a gas phase Beckmann rearrangement reaction which uses ethanol as a solvent, such that the overall process has higher economic efficiency (including the advantages of reducing the types of side reactions, lowering the amount of byproducts, and being more beneficial to separation, purification and refining of caprolactam), and the effects are obvious; on the other hand, compared with the practice in the prior art that the filtrate obtained by filtering is directly discharged during the catalyst preparation process, the present disclosure can apply the ethanol recovered from the catalyst preparation process in the gas phase Beckmann rearrangement reaction by using the ethanol as a reaction solvent, such an arrangement not only improves the caprolactam selectivity, but also reduces the production cost and alleviates the environmental protection pressure.

The present disclosure will be described in detail below with reference to examples.

Unless otherwise specified in the following examples, the pressure is a gauge pressure; the normal pressure means an atmospheric pressure; the normal temperature refers to 25° C.;

In the present disclosure, the content of the metallic element was measured by using an inductively coupled plasma (ICP) atomic emission spectrometer 7000DV, manufactured by PE (Perkin Elmer Incorporation) of the USA, under the test conditions as follows: the molecular sieve was dissolved by using HF acid to completely dissolve silicon oxide and metal oxide in the sample, and the content of metal ions was measured in an aqueous solution;

The external specific surface area and BET specific surface area of the molecular sieve were measured by an automatic adsorption apparatus with a model number ASAP-2460 manufactured by the Micromeritics Instrument Corporation in the USA, under the following test conditions: N₂ was used as an adsorbate, the adsorption temperature was −196.15° C. (liquid nitrogen temperature), and degassing was performed at 1.3 Pa and the constant temperature 300° C. for 6 hours;

The X-ray diffraction spectrum was recorded by a Miniflex600 type diffractometer manufactured by the Rigaku Corporation in Japan, and the test conditions were as follows: Cu target Ka radiation, Ni optical filter, the tube voltage was 40 kV, the tube current was 40 mA;

The prepared sample was analyzed by a field emission scanning electron microscope with a model number S-4800 manufactured by the Hitachi Corporation of Japan;

The molecular sieve and catalyst were respectively subjected to crystalline grain and particle size determinations on a FEI Tecnai G2F 20 field emission transmission electron microscope. A sample was prepared by adopting a suspension method, a catalyst sample was dispersed by using absolute ethanol, and vibrated uniformly, a small amount of dilute ink-shaped mixture was absorbed and dripped on a copper net, and the size of crystal grains or particles in the sample was observed after the ethanol is completely volatilized;

The abrasion index K of the catalyst was measured on an abrasion index analyzer according to the Industry Standard RIPP29-90 in the Petrochemical Analysis Method (Cuiding Yang, et al, Science Press of China, 1990) compiled by the SINOPEC Research Institute of Petroleum Processing (RIPP);

The particle size and particle size distribution of the catalyst were measured by a 2000E type laser particle size analyzer manufactured by the Dandong Bettersize Instruments Co., Ltd., the test method was a wet process test, water was used as a medium, and the mass concentration of a sample was within a range of 0.5%-2%, the scanning speed was 2,000 times/second;

The mist spray forming was carried out in a mist spray forming apparatus with a model number LT-300 manufactured by the Wuxi Tianyang Spray Drying Equipment Co., Ltd.;

In the following examples, washing was carried out with water until the pH of the filtered wash water was within a range of 9-10.5.

### Example 1

The catalyst was prepared according to the method provided by the present disclosure, the specific steps were as follows:

(1) 482 kg of ethanol with a content of 95 wt % and 302 kg of tetrapropylammonium hydroxide aqueous solution with a content of 22.5 wt % were respectively added into a stainless steel reaction kettle having a volume of 2 m³, the ingredients were stirred, 347 kg of tetraethoxysilane was then supplemented, the stirring process was continued, 332 kg of water and 38.6 g of Fe(NO₃)₃.9H₂O were further added, and the stirring process was continued for 4 hours under the normal temperature, so that a colloid mixture was obtained; wherein the molar ratio of tetraethoxysilane calculated by SiO₂:ethanol:tetrapropylammonium hydroxide:water was 1:10:0.2:20; the weight ratio of tetraethoxysilane calculated by SiO₂ relative to the metal source calculated by metallic element was 18666:1;

(2) The colloid mixture was subjected to crystallization with an ethanol-hydrothermal system, wherein the crystallization conditions comprising: crystallization was initially performed at 70° C. for 1 day, and crystallization was then performed at 100° C. for 2 days, such that the crystallization mother liquor with a pH of 13.51 was obtained;

(3) The obtained crystallization mother liquor was subjected to evaporating at 85° C. for 10 hours to evaporate ethanol (water was supplemented continuously during the process, so as to maintain the material at a certain liquid level, and recover water-containing ethanol solution for use); then a 50 nm six-tube membrane was used for washing and concentrating the crystallization mother liquor, the washing was performed by means of water with a temperature of 40-60° C., wherein the used amount of the washing water was 6.0 m³, and the washing process was continued until a pH of the washing water of the crystallized product reached 9.1. Following the washing and concentrating process, 395 kg of molecular sieve slurry with a solid content of 26.8 wt % was obtained.

A small amount of the molecular sieve slurry was taken and subjected to drying at 120° C. for 20 hours, the molecular sieve slurry was then subjected to roasting at 550° C. for 6 hours to produce the molecular sieve, wherein the molecular sieve had a content of metal element of 51.5 μg/g, a BET specific surface area of 426 m²/g, and an external specific surface area of 44 m²/g;

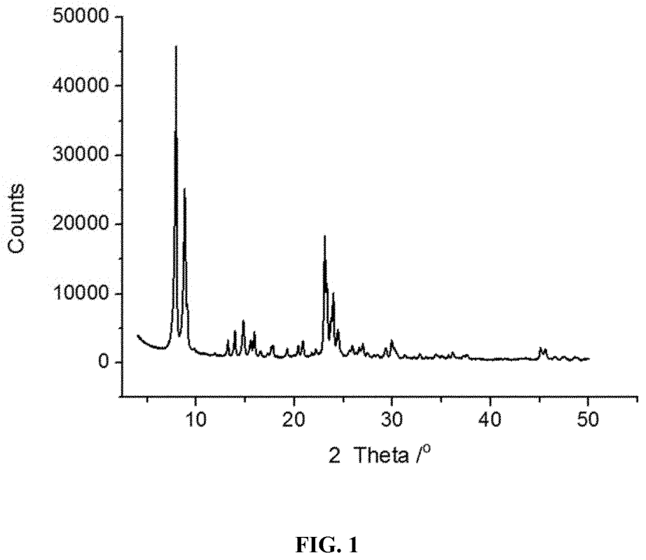

The X-ray diffraction spectrogram of the molecular sieve was shown in FIG. 1, the X-ray diffraction (XRD) spectrogram was consistent with the characteristics of MFI structure standard XRD spectrogram recorded in Microporous Materials, Vol. 22, p 637, 1998, it demonstrated that the molecular sieve had the MFI crystal structure;

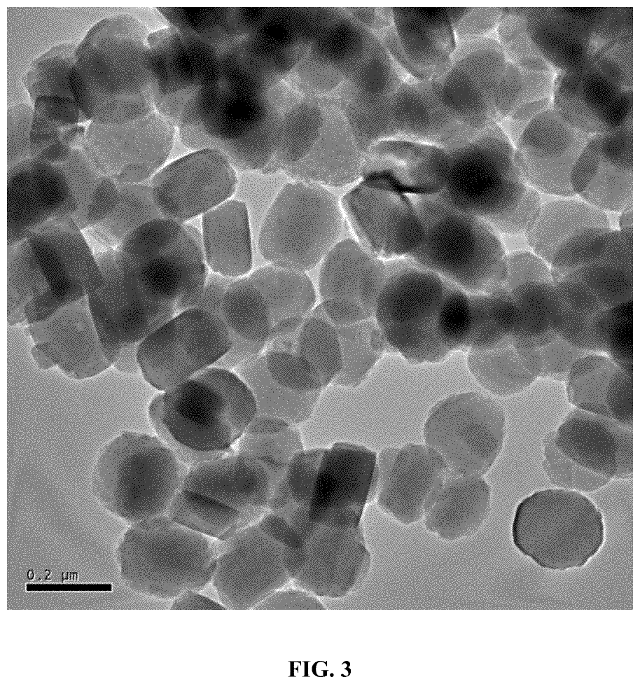

The transmission electron microscope (TEM) photograph was illustrated in FIG. 3, as can be seen from the TEM photograph that the MFI topological structure molecular sieve had uniform crystalline grain particle size and a particle size of 0.15-0.2 μm;

(4) The part of the molecular sieve slurry obtained in step (3) was mixed with 201 kg of alkaline silica sol with the content of 30 wt % (the pH was 9.5, the content of sodium ions was 324 ppm, the content of SiO₂ was 40 wt %, and the surface area of SiO₂ obtained after roasting was 225 m²/g), wherein the weight ratio of the molecular sieve based on the dry weight relative to the alkaline silica sol calculated by SiO₂ was 60:40, the mixture was stirred uniformly and pulped to obtain molecular sieve-binder slurry with the solid content of 25.2 wt %. The molecular sieve-binder slurry was conveyed to a mist spray forming device for performing the mist spray forming, wherein the inlet temperature and the outlet temperature of the mist spray forming device were 200° C. and 95° C. respectively. The mixture was then fed into a 3 m³ heating shuttle furnace (manufactured by Hubei Huanggang Huaxia Electromechanical Thermal Equipment Co., Ltd., hereinafter the same), and subjected to roasting at 280° C., 400° C. and 480° C. for 2 h respectively, and finally subjected to roasting at 550° C. for 12 h to obtain 149.5 kg microsphere molecular sieve, wherein the content of the MFI topological structure silicon molecular sieve containing a trace amount of metal ions with Lewis acid characteristic was 60 wt %, and the content of the binder calculated by SiO₂ was 40 wt %;

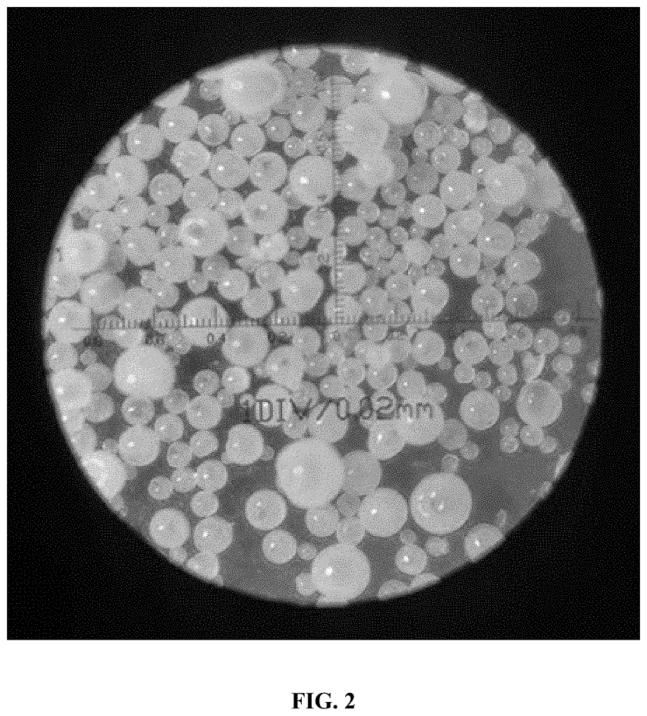

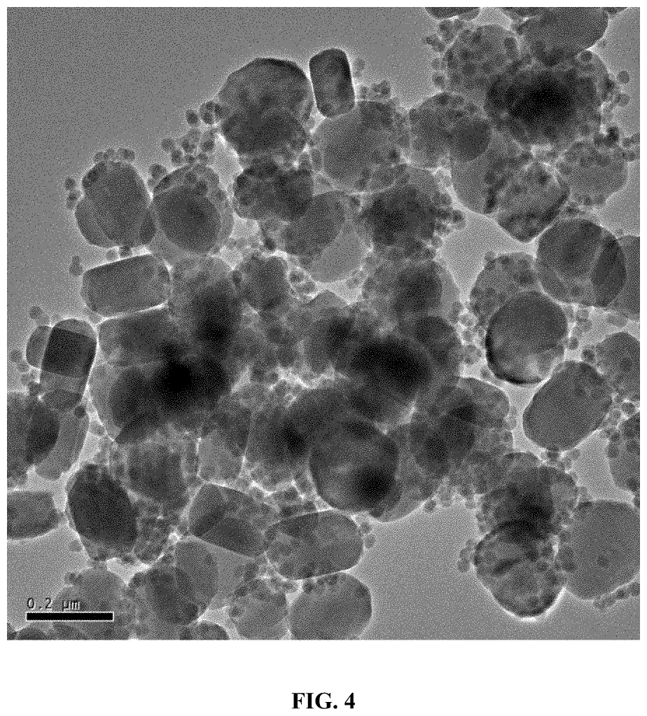

100 g of the microspherical molecular sieve and 1,000 g of an alkaline buffer solution of a nitrogen-containing compound (the alkaline buffer solution of a nitrogen-containing compound was a mixed solution of ammonia water and an ammonium nitrate aqueous solution, wherein the pH was 11.35, the content of the ammonia water was 26 wt %, the content of the ammonium nitrate in the ammonium nitrate aqueous solution was 7.5 wt %, the weight ratio of the ammonia water relative to the ammonium nitrate aqueous solution was 3:2) were added into a stainless steel reaction kettle having a volume of 2,000 ml (KCF-2 type magnetic stirring autoclave, manufactured by the Keli Automatic Control Equipment Research Institute of Yantai High-tech Zone, hereinafter the same), the mixture was subjected to stirring at a constant temperature of 86° C. and under a pressure of 2.7 kg/cm² for 2 hours, then subjected to filtering, and drying at 90° C. for 12 hours, the operations were then repeated once under the same conditions, and subjected to filtering, and washing until the pH of a filtration clear solution was about 9, and subsequently subjected to drying at 120° C. for 24 hours to prepare the catalyst S1. The photograph of catalyst S1 was shown in FIG. 2, and it can be seen from FIG. 2 that the particle size of the catalyst was very round and uniform; the TEM photograph of the catalyst S1 was illustrated in FIG. 4, the photograph showed that the tiny particles of 10-30 nm existed on the crystalline grains of the MFI topological structure all-silicon molecular sieve, and the tiny particles were silicon oxide binder.

The particle size distribution of the catalyst was shown in Table 1, the particle size of the catalyst was concentrated within a range of 70-200 μm, D₅₀=107.7 μm, and the abrasion index K was 1.2%/h.

TABLE 1

| Particle size μm | Range % | Accumulation % |
|---|---|---|
| 0.040-0.044 | 0.00 | 0 |
| 0.044-0.049 | 0.00 | 0 |
| 0.049-0.055 | 0.00 | 0 |
| 0.055-0.061 | 0.00 | 0 |
| 0.061-0.068 | 0.00 | 0 |
| 0.068-0.076 | 0.00 | 0 |
| 0.076-0.085 | 0.00 | 0 |
| 0.085-0.095 | 0.00 | 0 |
| 0.095-0.105 | 0.00 | 0 |
| 0.105-0.118 | 0.00 | 0 |
| 0.118-0.131 | 0.00 | 0 |
| 0.131-0.146 | 0.00 | 0 |
| 0.146-0.163 | 0.00 | 0 |
| 0.163-0.181 | 0.00 | 0 |
| 0.181-0.202 | 0.00 | 0 |
| 0.202-0.225 | 0.00 | 0 |
| 0.225-0.251 | 0.00 | 0 |
| 0.251-0.280 | 0.00 | 0 |
| 0.280-0.312 | 0.00 | 0 |
| 0.312-0.348 | 0.00 | 0 |
| 0.348-0.388 | 0.00 | 0 |
| 0.388-0.432 | 0.00 | 0 |
| 0.432-0.481 | 0.00 | 0 |
| 0.481-0.536 | 0.00 | 0 |
| 0.536-0.598 | 0.00 | 0 |
| 0.598-0.666 | 0.00 | 0 |
| 0.666-0.742 | 0.00 | 0 |
| 0.742-0.827 | 0.00 | 0 |
| 0.827-0.922 | 0.00 | 0 |
| 0.922-1.027 | 0.00 | 0 |
| 1.027-1.144 | 0.00 | 0 |
| 1.144-1.275 | 0.00 | 0 |
| 1.275-1.421 | 0.00 | 0 |
| 1.421-1.583 | 0.01 | 0.01 |
| 1.583-1.764 | 0.04 | 0.05 |
| 1.764-1.966 | 0.09 | 0.14 |
| 1.966-2.191 | 0.16 | 0.3 |
| 2.191-2.441 | 0.19 | 0.49 |
| 2.441-2.720 | 0.23 | 0.72 |
| 2.720-3.031 | 0.26 | 0.98 |
| 3.031-3.377 | 0.29 | 1.27 |
| 3.377-3.763 | 0.29 | 1.56 |
| 3.763-4.193 | 0.28 | 1.84 |
| 4.193-4.673 | 0.22 | 2.06 |
| 4.673-5.207 | 0.16 | 2.22 |
| 5.207-5.802 | 0.09 | 2.31 |
| 5.802-6.465 | 0.05 | 2.36 |
| 6.465-7.203 | 0.01 | 2.37 |
| 7.203-8.026 | 0.00 | 2.37 |
| 8.026-8.944 | 0.05 | 2.42 |
| 8.944-9.966 | 0.01 | 2.43 |
| 9.966-11.10 | 0.06 | 2.49 |
| 11.10-12.37 | 0.14 | 2.63 |
| 12.37-13.78 | 0.18 | 2.81 |
| 13.78-15.36 | 0.19 | 3 |
| 15.36-17.11 | 0.20 | 3.2 |
| 17.11-19.07 | 0.18 | 3.38 |
| 19.07-21.25 | 0.18 | 3.56 |
| 21.25-23.68 | 0.18 | 3.74 |
| 23.68-26.39 | 0.19 | 3.93 |
| 26.39-29.40 | 0.23 | 4.16 |
| 29.40-32.76 | 0.29 | 4.45 |
| 32.76-36.50 | 0.43 | 4.88 |
| 36.50-40.68 | 0.73 | 5.61 |
| 40.68-45.33 | 1.14 | 6.75 |
| 45.33-50.51 | 1.76 | 8.51 |
| 50.51-56.28 | 2.58 | 11.09 |
| 56.28-62.71 | 3.61 | 14.7 |
| 62.71-69.87 | 4.78 | 19.48 |
| 69.87-77.86 | 6.09 | 25.57 |
| 77.86-86.76 | 7.26 | 32.83 |
| 86.76-96.67 | 8.23 | 41.06 |
| 96.67-107.7 | 8.91 | 49.97 |
| 107.7-120.0 | 9.16 | 59.13 |
| 120.0-133.7 | 8.93 | 68.06 |
| 133.7-149.0 | 8.27 | 76.33 |
| 149.0-166.0 | 7.19 | 83.52 |
| 166.0-185.0 | 5.84 | 89.36 |
| 185.0-206.1 | 4.38 | 93.74 |
| 206.1-229.7 | 2.94 | 96.68 |
| 229.7-255.9 | 1.80 | 98.48 |
| 255.9-285.2 | 1.02 | 99.5 |
| 285.2-317.8 | 0.40 | 99.9 |
| 317.8-354.1 | 0.10 | 100 |
| 354.1-394.6 | 0.00 | 100 |
| 394.6-439.7 | 0.00 | 100 |
| 439.7-489.9 | 0.00 | 100 |
| 489.9-545.9 | 0.00 | 100 |
| 545.9-608.3 | 0.00 | 100 |
| 608.3-677.8 | 0.00 | 100 |
| 677.8-755.3 | 0.00 | 100 |
| 755.3-841.6 | 0.00 | 100 |
| 841.6-937.7 | 0.00 | 100 |
| 937.7-1044 | 0.00 | 100 |
| 1044-1164 | 0.00 | 100 |
| 1164-1297 | 0.00 | 100 |
| 1297-1445 | 0.00 | 100 |
| 1445-1610 | 0.00 | 100 |
| 1610-1794 | 0.00 | 100 |
| 1794-2000 | 0.00 | 100 |

### Example 2

The catalyst was prepared according to the method provided by the present disclosure, the specific steps were as follows:

(1) 810 kg of ethanol with a content of 95 wt % and 305 kg of tetrapropylammonium hydroxide aqueous solution with a content of 22.5 wt % were respectively added into a stainless steel reaction kettle having a volume of 2 m³, the ingredients were stirred, 347 kg of tetraethoxysilane was then supplemented, the stirring process was continued, 325 kg of water and 58.39 g of Al(NO₃)₃.9H₂O were further added, and the stirring process was continued for 4 hours under the normal temperature, so that a colloid mixture was obtained; wherein the molar ratio of tetraethoxysilane calculated by SiO₂:ethanol:tetrapropylammonium hydroxide:water was 1:14:0.2:20; the weight ratio of tetraethoxysilane calculated by SiO₂ relative to the metal source calculated by metallic element was 23700:1;

(2) The colloid mixture was subjected to crystallization with an ethanol-hydrothermal system, wherein the crystallization conditions comprising: crystallization was initially performed at 80° C. for 1 day, and crystallization was then performed at 100° C. for 2 days, such that the crystallization mother liquor with a pH of 13.68 was obtained;

(3) The obtained crystallization mother liquor was subjected to evaporating at 88° C. for 10 hours to evaporate ethanol (water was supplemented continuously during the process, so as to maintain the material at a certain liquid level, and recover water-containing ethanol solution for use); then a 50 nm six-tube membrane was used for washing and concentrating the crystallization mother liquor, the washing was performed by means of water with a temperature of 40-60° C., wherein the used amount of the washing water was 6.0 m³, and the washing process was continued until a pH of the washing water of the crystallized product reached 9.1. Following the washing and concentrating process, 436 kg of molecular sieve slurry with a solid content of 24.5 wt % was obtained.

A small amount of the molecular sieve slurry was taken and subjected to drying at 120° C. for 20 hours, the molecular sieve slurry was then subjected to roasting at 550° C. for 6 hours to produce the molecular sieve, wherein the molecular sieve had a content of metal element of 41.4 μg/g, a BET specific surface area of 425 m²/g, and an external specific surface area of 41 m²/g;

The X-ray diffraction (XRD) spectrogram of the molecular sieve was consistent with the characteristics of MFI structure standard XRD spectrogram recorded in Microporous Materials, Vol. 22, p 637, 1998, it demonstrated that the molecular sieve had the MFI crystal structure;

The transmission electron microscope (TEM) photograph illustrated that the MFI topological structure molecular sieve had uniform crystalline grain particle size and a particle size of 0.1-0.2 μm;

(4) The part of the molecular sieve slurry obtained in step (3) was mixed with 304 kg of alkaline silica sol with the content of 30 wt % (the pH was 9.5, the content of sodium ions was 324 ppm, the content of SiO₂ was 40 wt %, and the surface area of SiO₂ obtained after roasting was 225 m²/g), wherein the weight ratio of the molecular sieve based on the dry weight relative to the alkaline silica sol calculated by SiO₂ was 50:50, then 60 kg of water was added, the mixture was stirred uniformly and pulped to obtain molecular sieve-binder slurry with the solid content of 22.5 wt %. The molecular sieve-binder slurry was conveyed to a mist spray forming device for performing the mist spray forming, wherein the inlet temperature and the outlet temperature of the mist spray forming device were 205° C. and 100° C. respectively. The mixture was then fed into a 3 m³ heating shuttle furnace, and subjected to roasting at 280° C., 400° C. and 480° C. for 2 h respectively, and finally subjected to roasting at 550° C. for 12 h to obtain 181.5 kg microsphere molecular sieve, wherein the content of the MFI topological structure silicon molecular sieve containing a trace amount of metal ions with Lewis acid characteristic was 50 wt %, and the content of the binder calculated by SiO₂ was 50 wt %;

95 g of the microspherical molecular sieve and 950 g of an alkaline buffer solution of a nitrogen-containing compound (the alkaline buffer solution of a nitrogen-containing compound was a mixed solution of ammonia water and an ammonium nitrate aqueous solution, wherein the pH was 11.39, the content of the ammonia water was 26 wt %, the content of the ammonium acetate in the ammonium acetate aqueous solution was 7.5 wt %, the weight ratio of the ammonia water relative to the ammonium acetate aqueous solution was 3:2) were added into a stainless steel reaction kettle having a volume of 2,000 ml, the mixture was subjected to stirring at a constant temperature of 85° C. and under a pressure of 2.6 kg/cm² for 2 hours, then subjected to filtering, and drying at 90° C. for 12 hours, the operations were then repeated once under the same conditions, and subjected to filtering, and washing until the pH of a filtration clear solution was about 9, and subsequently subjected to drying at 120° C. for 24 hours to prepare the catalyst S2;

The particle size of the catalyst was concentrated within a range of 75-150 μm, and the abrasion index K was 1.4%/h.

### Example 3

The catalyst was prepared according to the method provided by the present disclosure, the specific steps were as follows:

(1) 725 kg of ethanol with a content of 95 wt % and 302 kg of tetrapropylammonium hydroxide aqueous solution with a content of 22.5 wt % were respectively added into a stainless steel reaction kettle having a volume of 2 m³, the ingredients were stirred, 347 kg of tetraethoxysilane was then supplemented, the stirring process was continued, 330 kg of water and 37.37 g of Cr(NO₃)₃.9H₂O were further added, and the stirring process was continued for 4 hours under the normal temperature, so that a colloid mixture was obtained; wherein the molar ratio of tetraethoxysilane calculated by SiO₂:ethanol:tetrapropylammonium hydroxide:water was 1:13:0.2:20; the weight ratio of tetraethoxysilane calculated by SiO₂ relative to the metal source calculated by metallic element was 20600:1;

(2) The colloid mixture was subjected to crystallization with an ethanol-hydrothermal system, wherein the crystallization conditions comprising: crystallization was initially performed at 65° C. for 1 day, and crystallization was then performed at 120° C. for 2 days, such that the crystallization mother liquor with a pH of 13.54 was obtained;

(3) The obtained crystallization mother liquor was subjected to evaporating at 85° C. for 10 hours to evaporate ethanol (water was supplemented continuously during the process, so as to maintain the material at a certain liquid level, and recover water-containing ethanol solution for use); then a 50 nm six-tube membrane was used for washing and concentrating the crystallization mother liquor, the washing was performed by means of water with a temperature of 40-60° C., wherein the used amount of the washing water was 6.5 m³, and the washing process was continued until a pH of the washing water of the crystallized product reached 9. Following the washing and concentrating process, 375 kg of molecular sieve slurry with a solid content of 28.4 wt % was obtained.

A small amount of the molecular sieve slurry was taken and subjected to drying at 120° C. for 20 hours, the molecular sieve slurry was then subjected to roasting at 550° C. for 6 hours to produce the molecular sieve, wherein the molecular sieve had a content of metal element of 46.8 μg/g, a BET specific surface area of 435 m²/g, and an external specific surface area of 46 m²/g;

The X-ray diffraction (XRD) spectrogram of the molecular sieve was consistent with the characteristics of MFI structure standard XRD spectrogram recorded in Microporous Materials, Vol. 22, p 637, 1998, it demonstrated that the molecular sieve had the MFI crystal structure;

The transmission electron microscope (TEM) photograph illustrated that the MFI topological structure molecular sieve had uniform crystalline grain particle size and a particle size of 0.1-0.2 μm;

(4) The part of the molecular sieve slurry obtained in step (3) was mixed with 96 kg of alkaline silica sol with the content of 30 wt % (the pH was 9.5, the content of sodium ions was 324 ppm, the content of SiO₂ was 40 wt %, and the surface area of SiO₂ obtained after roasting was 225 m²/g), wherein the weight ratio of the molecular sieve based on the dry weight relative to the alkaline silica sol calculated by SiO₂ was 76:24, then 330 kg of water was added, the mixture was stirred uniformly and pulped to obtain molecular sieve-binder slurry with the solid content of 15 wt %. The molecular sieve-binder slurry was conveyed to a mist spray forming device for performing the mist spray forming, wherein the inlet temperature and the outlet temperature of the mist spray forming device were 210° C. and 105° C. respectively. The mixture was then fed into a 3 m³ heating shuttle furnace, and subjected to roasting at 280° C., 400° C. and 480° C. for 2 h respectively, and finally subjected to roasting at 550° C. for 12 h to obtain 119.8 kg microsphere molecular sieve, wherein the content of the MFI topological structure silicon molecular sieve containing a trace amount of metal ions with Lewis acid characteristic was 76 wt %, and the content of the binder calculated by SiO₂ was 24 wt %;

100 g of the microspherical molecular sieve and 1,000 g of an alkaline buffer solution of a nitrogen-containing compound (the alkaline buffer solution of a nitrogen-containing compound was a mixed solution of ammonia water and an ammonium nitrate aqueous solution, wherein the pH was 11.35, the content of the ammonia water was 26 wt %, the content of the ammonium nitrate in the ammonium nitrate aqueous solution was 7.5 wt %, the weight ratio of the ammonia water relative to the ammonium nitrate aqueous solution was 3:2) were added into a stainless steel reaction kettle having a volume of 2,000 ml, the mixture was subjected to stirring at a constant temperature of 90° C. and under a pressure of 3.2 kg/cm² for 2 hours, then subjected to filtering, and drying at 90° C. for 12 hours, the operations were then repeated once under the same conditions, and subjected to filtering, and washing until the pH of a filtration clear solution was about 9, and subsequently subjected to drying at 120° C. for 24 hours to prepare the catalyst S3;

The particle size of the catalyst was concentrated within a range of 55-120 μm, and the abrasion index K was 2.8%/h.

### Example 4

The catalyst was prepared according to the method provided by the present disclosure, the specific steps were as follows:

(1) 725 kg of ethanol with a content of 95 wt % and 305 kg of tetrapropylammonium hydroxide aqueous solution with a content of 22.5 wt % were respectively added into a stainless steel reaction kettle having a volume of 2 m³, the ingredients were stirred, 347 kg of tetraethoxysilane was then supplemented, the stirring process was continued, 330 kg of water and 12.1 g of Ce(NO₃)₃.7H₂O were further added, and the stirring process was continued for 4 hours under the normal temperature, so that a colloid mixture was obtained; wherein the molar ratio of tetraethoxysilane calculated by SiO₂:ethanol:tetrapropylammonium hydroxide:water was 1:13:0.2:20; the weight ratio of tetraethoxysilane calculated by SiO₂ relative to the metal source calculated by metallic element was 26700:1;

(2) The colloid mixture was conveyed to a reaction kettle and subjected to crystallization with an ethanol-hydrothermal system, wherein the crystallization conditions comprising: crystallization was initially performed at 65° C. for 1 day, and crystallization was then performed at 120° C. for 2 days, such that the crystallization mother liquor with a pH of 13.55 was obtained;

(3) The obtained crystallization mother liquor was subjected to evaporating at 85° C. for 10 hours to evaporate ethanol (water was supplemented continuously during the process, so as to maintain the material at a certain liquid level, and recover water-containing ethanol solution for use); then a 50 nm six-tube membrane was used for washing and concentrating the crystallization mother liquor, the washing was performed by means of water with a temperature of 40-60° C., wherein the used amount of the washing water was 6.5 m³, and the washing process was continued until a pH of the washing water of the crystallized product reached 9. Following the washing and concentrating process, 452 kg of molecular sieve slurry with a solid content of 23.4 wt % was obtained.

A small amount of the molecular sieve slurry was taken and subjected to drying at 120° C. for 20 hours, the molecular sieve slurry was then subjected to roasting at 550° C. for 6 hours to produce the molecular sieve, wherein the molecular sieve had a content of metal element of 36.6 μg/g, a BET specific surface area of 431 m²/g, and an external specific surface area of 49 m²/g;

The X-ray diffraction (XRD) spectrogram of the molecular sieve was consistent with the characteristics of MFI structure standard XRD spectrogram recorded in Microporous Materials, Vol. 22, p 637, 1998, it demonstrated that the molecular sieve had the MFI crystal structure;

The transmission electron microscope (TEM) photograph illustrated that the MFI topological structure molecular sieve had uniform crystalline grain particle size and a particle size of 0.1-0.2 μm;

(4) The part of the molecular sieve slurry obtained in step (3) was mixed with 129 kg of alkaline silica sol with the content of 30 wt % (the pH was 9.5, the content of sodium ions was 324 ppm, the content of SiO₂ was 40 wt %, and the surface area of SiO₂ obtained after roasting was 225 m²/g), wherein the weight ratio of the molecular sieve based on the dry weight relative to the alkaline silica sol calculated by SiO₂ was 70:30, then 10 kg of water was added, the mixture was stirred uniformly and pulped to obtain molecular sieve-binder slurry with the solid content of 24.5 wt %. The molecular sieve-binder slurry was conveyed to a mist spray forming device for performing the mist spray forming, wherein the inlet temperature and the outlet temperature of the mist spray forming device were 200° C. and 95° C. respectively. The mixture was then fed into a 3 m³ heating shuttle furnace, and subjected to roasting at 280° C., 400° C. and 480° C. for 2 h respectively, and finally subjected to roasting at 550° C. for 12 h to obtain 142.7 kg microsphere molecular sieve, wherein the content of the MFI topological structure silicon molecular sieve containing a trace amount of metal ions with Lewis acid characteristic was 70 wt %, and the content of the binder calculated by SiO₂ was 30 wt %;

100 g of the microspherical molecular sieve and 1,000 g of an alkaline buffer solution of a nitrogen-containing compound (the alkaline buffer solution of a nitrogen-containing compound was a mixed solution of ammonia water and an ammonium nitrate aqueous solution, wherein the pH was 11.35, the content of the ammonia water was 26 wt %, the content of the ammonium nitrate in the ammonium nitrate aqueous solution was 7.5 wt %, the weight ratio of the ammonia water relative to the ammonium nitrate aqueous solution was 3:2) were added into a stainless steel reaction kettle having a volume of 2,000 ml, the mixture was subjected to stirring at a constant temperature of 82° C. and under a pressure of 2.3 kg/cm² for 2 hours, then subjected to filtering, and drying at 90° C. for 12 hours, the operations were then repeated once under the same conditions, and subjected to filtering, and washing until the pH of a filtration clear solution was about 9, and subsequently subjected to drying at 120° C. for 24 hours to prepare the catalyst S4;

The particle size of the catalyst was concentrated within a range of 70-150 μm, and the abrasion index K was 2%/h.

### Example 5

The catalyst was prepared according to the same method as in Example 1, except that the metal source was replaced with tetrabutyltitanate, and the weight ratio of tetraethoxysilane calculated by SiO₂ relative to the metal source calculated by metallic element was 50000:1.

The obtained molecular sieve had a content of metal element of 19.2 μg/g, a BET specific surface area of 448 m²/g, and an external specific surface area of 46 m²/g;

The catalyst S5 was prepared, the particle size of the catalyst was concentrated within a range of 70-150 μm, and the abrasion index K was 1.8%/h.

### Example 6

The catalyst was prepared according to the same method as in Example 1, except that in step (1), 95 wt % of ethanol was used in an amount of 194 kg and water was used in an amount of 366 kg;

The molar ratio of tetraethoxysilane calculated by SiO₂:ethanol:tetrapropylammonium hydroxide:water was 1:6.4:0.2:20;

The catalyst S6 was prepared, the particle size of the catalyst was concentrated within a range of 60-150 μm, and the abrasion index K was 1.8%/h.

### Comparative Example 1

The catalyst was prepared according to the same method as in Example 1, except that in the step (2), the conditions of crystallization with an ethanol-hydrothermal system were as follows: crystallization was performed by an ethanol-hydrothermal system at 100° C. for 3 days;

The catalyst D1 was prepared, the particle size of the catalyst was within a range of 70-150 μm, and the abrasion index K was 2.0%/h.

### Comparative Example 2

The catalyst was prepared according to the same method as in Example 1, except that Fe(NO₃)₃.9H₂O was not added in the step (1);

The catalyst D2 was prepared, the particle size of the catalyst was within a range of 70-150 μm, and the abrasion index K was 2.1%/h.

### Comparative Example 3

The catalyst was prepared according to the same method as in Example 1, except that in the step (1), Fe(NO₃)₃.9H₂O was used in an amount of 96.5 g, the weight ratio of tetraethoxysilane calculated by SiO₂ relative to the metal source calculated by metallic element was 7500:1;

The catalyst D3 was prepared, the particle size of the catalyst was within a range of 70-150 μm, and the abrasion index K was 1.7%/h.

### Test Example 1

The test example was used for evaluating the catalytic reaction effect of the catalyst containing the MFI topological structure molecular sieve prepared in the examples and the comparative examples in the gas phase Beckmann rearrangement reaction:

The gas phase Beckmann rearrangement reaction of cyclohexanone oxime was performed in a fixed bed reactor, the inner diameter of the reactor was 5 mm, 0.375 g of 40-60 mesh catalyst calculated by the molecular sieve was filled in the reactor, coarse quartz sand with the height of about 30 mm and the size of 30 meshes was filled above the catalyst bed layer, and fine quartz sand with the size of 50 meshes was filled underneath the catalyst bed layer. The rearrangement reaction conditions comprising: the pressure was normal pressure; the reaction temperature was 380° C.; the cyclohexanone oxime weight hourly space velocity (WHSV, the flow rate of cyclohexanone oxime in the feeding materials/the weight of catalyst calculated by a molecular sieve in a bed layer) was 16 h⁻¹; the reaction solvent was ethanol, and the weight of the ethanol was 65 wt % of the reaction raw materials; the flow rate of the carrier gas (N₂) was 45 mL/min, the reaction product was cooled by an ice-water mixture and then entered a collecting bottle for gas-liquid separation, and the composition analysis of the product was implemented after the reaction was carried out for 6 hours.

The reaction product was quantitatively analyzed by a gas chromatograph (hydrogen flame ion detector, PEG20M capillary chromatographic column, column length 50 m) with a model number 6890 manufactured by the Agilent Technologies Company, the vaporization chamber temperature was 250° C., the detection chamber temperature was 240° C., the column temperature was subjected to the programmed temperature rise, the temperature was maintained at 110° C. for 8 minutes, and then increased to 230° C. at the temperature rise rate of 15° C./min, the temperature was subsequently maintained at 230° C. for 14 minutes.

The content of rearrangement products of caprolactam and cyclohexanone oxime after the reaction was calculated by adopting an area normalization method, and the solvent did not participate in the integration calculation.

The molar percentage content of cyclohexanone oxime in the reaction product and the molar percentage content of caprolactam in the reaction product were obtained through the analysis, and the conversion rate of the cyclohexanone oxime and the selectivity of the caprolactam were calculated through the following equations:

Conversion rate of the cyclohexanone oxime(mol %)=(molar content of cyclohexanone oxime in the feeding materials−molar content of cyclohexanone oxime in the product)/molar content of cyclohexanone oxime in the feeding materials×100%

Total selectivity of Caprolactam(mol %)=(molar content of caprolactam in product/(100−molar content of cyclohexanone oxime in the product)×100%

The Ethyl-Epsilon-Caprolactam (AEH) was present in the by-products of the gas phase Beckmann rearrangement reaction of cyclohexanone oxime by an amount of about 40%, the by-products was formed by the alcoholysis reaction of ethanol with the enol-structure tautomer of caprolactam. Under the action of water, the Ethyl-Epsilon-Caprolactam continuously generated caprolactam through hydrolysis reaction. Thus, the total selectivity of caprolactam was calculated by including the amount of caprolactam generated from hydrolysis of Ethyl-Epsilon-Caprolactam; the results were as shown in Table 2.

TABLE 2

| | Conversion | | | |
|---|---|---|---|---|
| | rate of | | | Total |
| | cyclohexanone | Selectivity | Selectivity | selectivity of |
| | oxime, | of CPL, | of AEH, | caprolactam, |
| | mol % | mol % | mol % | mol % |
| Example 1 | 99.66 | 95.40 | 2.16 | 97.13 |
| Example 2 | 99.53 | 95.46 | 2.22 | 97.24 |
| Example 3 | 99.35 | 95.32 | 2.09 | 96.99 |
| Example 4 | 99.42 | 95.48 | 2.28 | 97.30 |
| Example 5 | 99.12 | 95.44 | 2.17 | 97.18 |
| Example 6 | 99.59 | 94.41 | 2.36 | 96.30 |
| Comparative | 98.65 | 95.10 | 2.06 | 96.75 |
| Example 1 | | | | |
| Comparative | 98.61 | 95.42 | 2.03 | 97.01 |
| Example 2 | | | | |
| Comparative | 99.44 | 94.15 | 2.04 | 95.78 |
| Example 3 | | | | |

Note:
CPL represents caprolactam; AEH represents ethyl-epsilon-caprolactam.

The results of Table 2 demonstrate that the catalyst containing the MFI topological structure silicon molecular sieve obtained through the preparation method provided by the present disclosure has better performance, the conversion rate of cyclohexanone oxime in the gas phase Beckmann rearrangement reaction of cyclohexanone oxime is higher and may reach up to 99.66%, the total selectivity of caprolactam is higher and may reach up to 97.30%, thus the effects are significant.

In addition, the catalyst provided by the present disclosure has lower abrasion index under the preferable circumstance, and is particularly suitable for a process for preparing caprolactam with the gas phase Beckmann rearrangement reaction of cyclohexanone oxime.

The above content describes in detail the preferred embodiments of the present disclosure, but the present disclosure is not limited thereto. A variety of simple modifications can be made in regard to the technical solutions of the present disclosure within the scope of the technical concept of the present disclosure, including a combination of individual technical features in any other suitable manner, such simple modifications and combinations thereof shall also be regarded as the content disclosed by the present disclosure, each of them falls into the protection scope of the present disclosure.

1. A MFI topological structure silicon molecular sieve comprising a silicon element, an oxygen element and a metallic element, wherein the ions of said metallic element have a Lewis acid characteristic; the content of the metallic element in the molecular sieve is within a range of 5-100 μg/g based on the total amount of the molecular sieve; the BET specific surface area of the molecular sieve is within a range of 400-500 m²/g.

2. The molecular sieve of claim 1, wherein the metallic element is at least one selected from the group consisting of transition metallic element, group IIIA element and group IVA element; the transition metallic element is at least one selected from the group consisting of group IB, group IIB, group IVB, group VB, group VIB, group VIIB and group VIII; and/or
the content of the metallic element in the molecular sieve is within a range of 6-90 μg/g, based on the total amount of the molecular sieve.

3. The molecular sieve of claim 1, wherein the metallic element is at least one selected from the group consisting of Al, Ga, Ge, Ce, Ag, Co, Ni, Cu, Zn, Mn, Pd, Pt, Cr, Fe, Au, Ru, Rh, Ti, Zr, V, Mo and W; and/or the molecular sieve has a BET specific surface area within a range of 420-450 m²/g, a crystalline grain particle size within a range of 0.1-0.3 μm, and an external specific surface area within a range of 30-60 m²/g.

4. The molecular sieve of claim 1, wherein the metallic element has an ionic valence state of +3 and/or an ionic valence state of +4.

5. A method for preparing a MFI topological structure silicon molecular sieve comprises the following steps:
(1) mixing ethyl orthosilicate, ethanol, metal source, tetrapropylammonium hydroxide with water to obtain a colloid mixture; wherein the molar ratio of ethyl orthosilicate calculated by SiO₂, ethanol, tetrapropylammonium hydroxide and water is 1:(4-25):(0.06-0.45):(6-100); the weight ratio of the ethyl orthosilicate calculated by SiO₂ relative to the metal source calculated by metallic element is (10,000-200,000):1;
(2) subjecting the colloid mixture to a two-stage crystallization with an ethanol-hydrothermal system under variable temperatures, wherein the conditions of the two-stage crystallization with an ethanol-hydrothermal system under variable temperatures comprise: crystallizing at 40-80° C. for 0.5-5 days, and then crystallizing at 80-130° C. for 0.5-5 days;
(3) subjecting the crystallization mother liquor obtained in the step (2) to filtering and roasting sequentially to obtain a molecular sieve;
the ions of the metallic element in the metal source have a Lewis acid characteristic.

6. The method of claim 5, wherein the molar ratio of ethyl orthosilicate calculated by SiO₂, ethanol, tetrapropylammonium hydroxide and water is 1:(4-15):(0.06-0.3):(15-50); and/or
the weight ratio of the ethyl orthosilicate calculated by SiO₂ relative to the metal source calculated by metallic element is (10000-100000): 1; and/or
the metal source is at least one selected from the group consisting of a metal nitrate, a metal chloride, a metal sulfate, a metal acetate, and an ester metal compound.

7. The method of claim 5, wherein the metallic element is at least one selected from the group consisting of transition metallic element, group IIIA element and group IVA element;
the transition metallic element is at least one selected from the group consisting of group IB, group IIB, group IVB, group VB, group VIB, group VIIB and group VIII.

8. The method of claim 5, wherein the metallic element is at least one element selected from the group consisting of Al, Ga, Ge, Ce, Ag, Co, Ni, Cu, Zn, Mn, Pd, Pt, Cr, Fe, Au, Ru, Rh, Ti, Zr, V, Mo and W.

9. The method of claim 5, wherein the metallic element has an ionic valence state of +3 and/or an ionic valence state of +4.

10. The method of claim 5, wherein the conditions of the two-stage crystallization with an ethanol-hydrothermal system under variable temperatures comprise: crystallizing at 50-80° C. for 1-1.5 days, and then crystallizing at 100-120° C. for 1-3 days.

11. The method of claim 5, wherein the method further comprises: the crystallization mother liquor is subjected to ethanol removal prior to the filtration in step (3).

12. The method of claim 11, wherein the conditions of ethanol removal comprise: the temperature is within a range of 50−90° C.; the time is within a range of 1-24 h.

13. The method of claim 5, wherein the roasting conditions comprise: the temperature is within a range of 400−600° C.; the time is within a range of 1-20 hours.

14. A catalyst comprising a silicon molecular sieve with a MFI topological structure silicon molecular sieve, wherein the catalyst comprising a molecular sieve and a binder; the content of the molecular sieve based on the dry weight in the catalyst is 50-95 wt %, and the content of the binder in terms of oxide is 5-50 wt %, based on the dry weight of the catalyst;
the molecular sieve comprises metallic element, the ions of the metallic element have a Lewis acid characteristic; the content of the metallic element in the molecular sieve is 5-100 μg/g based on the total amount of the molecular sieve.

15. The catalyst of claim 14, wherein the metallic element is at least one selected from the group consisting of transition metallic element, group IIIA element and group IVA element; the transition metallic element is preferably at least one metallic element selected from the group consisting of group IB, group IIB, group IVB, group VB, group VIB, group VIIB and group VIII; and/or
the content of the metallic element in the molecular sieve is within a range of 6-90 μg/g, based on the total amount of the molecular sieve.

16. The catalyst of claim 14, wherein the metallic element is at least one selected from the group consisting of Al, Ga, Ge, Ce, Ag, Co, Ni, Cu, Zn, Mn, Pd, Pt, Cr, Fe, Au, Ru, Rh, Ti, Zr, V, Mo and W; and/or
the molecular sieve has a BET specific surface area within a range of 420-450 m²/g, a crystalline grain particle size within a range of 0.1-0.3 μm, and an external specific surface area within a range of 30-60 m²/g.

17. The catalyst of claim 14, wherein the metallic element has an ionic valence state of +3 and/or an ionic valence state of +4.

18. The catalyst of claim 14, wherein the particle size of the catalyst is within a range of 20-200 μm; and/or
the catalyst has an abrasion index K less than 3%/h.

19. The catalyst of claim 14, wherein the content of the molecular sieve based on the dry weight in the catalyst is 50-70 wt %, and the content of the binder in terms of oxide is 30-50 wt %, based on the dry weight of the catalyst.

20. The catalyst of claim 14, wherein the binder is silicon oxide.
